(19) United States
(12) Patent Application Publication
(10) Pub. No.: US 2020/0158246 A1
(43) Pub. Date: May 21, 2020

(54) Pressure Regulating Valve for Cupping Negative Pressure Apparatus

(71) Applicants: Po-Chang Liu, Taoyuan City (TW); Biboting International Co., Ltd., Taoyuan City (TW)

(72) Inventors: Po-Chang Liu, Taoyuan City (TW); Shih-Ta Liu, Taoyuan City (TW)

(21) Appl. No.: 16/673,986

(22) Filed: Nov. 5, 2019

(30) Foreign Application Priority Data
Nov. 16, 2018 (TW) 107215570

(51) Int. Cl. F16K 3/26; G05D 16/04; F16K 47/02; B01D 39/18; A61M 1/08; A61M 1/00

(52) U.S. Cl. CPC F16K 3/26; G05D 16/04; F16K 47/02; B01D 46/24; A61M 1/08; A61M 1/0035; B01D 39/18

(57) ABSTRACT

A pressure regulating valve for a cupping negative pressure apparatus is provided with an upper pressure regulating valve screw cap and a lower pressure regulating valve body having a metal nut structure inside, and the metal nut structure is provided with a plurality of nut pressure relief holes. The pressure regulating valve screw cap can be tightened to block circulation of the nut pressure relief holes. A muffler cover assembly seat is disposed on an outermost side of a metal nut structure assembly seat for assembling a muffler cover, small holes for circulating air are disposed between the muffler cover and the metal nut structure assembly seat. The pressure regulating valve body has negative pressure communication holes for sucking and discharging air and the negative pressure communication holes are communicated or plugged by quick-plug screw caps. A filter cotton is disposed in the pressure regulating valve body.

### BACKGROUND OF THE INVENTION

### Field of Invention

The present invention belongs to the field of health care devices, and more particularly relates to a pressure regulating valve for adjusting a negative pressure force of a cupping negative pressure apparatus.

### Related Art

Cupping negative pressure apparatus is an easy-to-use health care device that has been widely promoted in the market. The cupping negative pressure apparatus is operated by connecting cupping cups with communicating tubes, an internal air pump of the apparatus is operated to achieve continuous suction vacuum effect, and a pressure relief valve releases a small amount of vacuum pressure when suction is stopped, so that the vacuum pressure inside the cupping cup produces actions of tightening and loosening, thus achieving massage effect on the human body.

Although the conventional cupping negative pressure apparatus has been able to achieve certain massage effect, in actual application, because different users need to feel different negative pressure forces and effects being achieved, the conventional cupping negative pressure apparatus can not meet demands of different users. Therefore, improvement must be made to obtain a way to adjust the negative pressure force to meet different preferences of users.

During the process of operation, the conventional cupping negative pressure apparatus usually has a plurality of negative pressure communication holes to connect to a plurality of communicating tubes and cupping cups simultaneously for negative pressure operation. When some of the negative pressure communication holes do not need to be operated, they are usually plugged with a soft plug, because the soft plug is not a structure of the conventional cupping negative pressure apparatus, it is easy to get lost during use or storage, and it takes time to obtain a substitute.

In addition, during the operation of negative pressure massage, since the user's body inevitably generates moisture or needs to be applied with essential oil, the conventional cupping negative pressure apparatus easily sucks in the substances and affects the suction force when performing suction and discharge action. Thus it is necessary to filter out the substances to maintain normal use of the apparatus.

Therefore, it can be seen that the conventional technology still has drawbacks and needs to be researched and improved by the industry.

### SUMMARY OF THE INVENTION

A main object of the present invention is to provide an effective method for adjusting a negative pressure force to meet the demands of different users, and to ensure the effects achieved by negative pressure massage.

Another object of the present invention is to provide a quick-plug screw cap that is mounted on a cupping negative pressure apparatus, which effectively controls opening and closing of negative pressure communication holes of the cupping negative pressure apparatus, and is less likely to be lost.

A further object of the present invention is that the cupping negative pressure apparatus effectively filters impurities during suction and discharge action to ensure the maintenance of suction force and further reduce malfunction of the apparatus.

A pressure regulating valve for a cupping negative pressure apparatus of the present invention is characterized in that a pressure regulating valve is disposed at a joint between communicating tubes and cupping cups of a cupping negative pressure apparatus, and the pressure regulating valve is divided into three components, including a pressure regulating valve screw cap, a pressure regulating valve body and a muffler cover.

The pressure regulating valve for the cupping negative pressure apparatus of the present invention is further improved in that the pressure regulating valve body is provided with a pressure regulating valve filter trough, a metal nut structure assembly seat is disposed above the pressure regulating valve filter trough, and the metal nut structure assembly seat is assembled integrally with a metal nut structure.

The metal nut structure assembly seat is provided with a metal nut structure placement hole for inserting and assembling the metal nut structure, and the metal nut structure placement hole and the metal nut structure are communicated with a plurality of small holes for allowing a small amount of air to flow to reach inside the pressure regulating valve body to reduce a negative pressure force.

The pressure regulating valve for the cupping negative pressure apparatus of the present invention is further improved in that outside the metal nut structure placement hole of the metal nut structure assembly seat is surrounded with a screw cap retaining ring, the screw cap retaining ring is provided with a plurality of small holes for allowing air to circulate to reach inside the pressure regulating valve body, and the screw cap retaining ring is capable of abutting against a bottom of a cap of the pressure regulating valve screw cap, so that the pressure regulating valve screw cap is capable of only rotating to a certain extent.

The pressure regulating valve for the cupping negative pressure apparatus of the present invention is further improved in that a bottom of the pressure regulating valve screw cap is provided with a metal screw structure for rotationally assembling with the metal nut structure inside the pressure regulating valve body.

The pressure regulating valve for the cupping negative pressure apparatus of the present invention is further improved in that the metal screw structure inside the pressure regulating valve screw cap and the metal nut structure inside the pressure regulating valve body can be driven to be rotationally connected to a tightest state by rotating the pressure regulating valve screw cap. In the tightest state, the metal screw structure is capable of blocking the small holes on the metal nut structure inside the pressure regulating valve body, so that air is unable to flow to the pressure regulating valve body, and thus the negative pressure force is unaffected to maintain in its strongest state. When the metal screw structure and the metal nut structure are rotationally loosened, the metal screw structure moves upward to expose the small holes of the metal nut structure with gaps, so that air flows through the gaps to reach inside the pressure regulating valve body, thereby reducing the negative pressure force, and controlling an amount of air flowing to reach inside the pressure regulating valve body by rotating the pressure regulating valve screw cap in order to adjust a magnitude of the negative pressure force.

The pressure regulating valve for the cupping negative pressure apparatus of the present invention is further improved in that an outermost ring of the metal nut structure assembly seat is provided with a ring of muffler cover assembly seat to be capped by the muffler cover. The muffler cover is provided with a hole, the muffler cover passes through a top of the pressure regulating valve screw cap via the hole to be assembled with the muffler cover assembly seat of the metal nut structure assembly seat, so that retaining ring circulation holes of the pressure regulating valve body are located in an internal space formed by assembling the muffler cover and the muffler cover assembly seat. Thereby, pressure is relieved in the internal space and noise generated by pressure relief is buffered, and air is slowly circulated from a gap underneath the muffler cover and the pressure regulating valve screw cap to reach inside the pressure regulating valve body to reduce the noise generated by pressure relief.

The pressure regulating valve for the cupping negative pressure apparatus of the present invention is further improved in that the pressure regulating valve body is provided with the negative pressure communication holes for functioning as air suction and discharge passages, and through the communicating tubes the pressure regulating valve of the present invention is connected in series between the negative pressure apparatus and negative pressure cupping cups, and therefore negative pressures in the communicating tubes are affected through the pressure regulating valve in order to control negative pressure forces inside the negative pressure cupping cups.

The pressure regulating valve for the cupping negative pressure apparatus of the present invention is further improved in that outer sides of the negative pressure communication holes of the pressure regulating valve body are sleeved with the quick-plug screw caps, and the unused negative pressure communication holes are plugged by the quick-plug screw caps to prevent air from flowing through the unused negative pressure communication holes, which will affect the negative pressure.

The pressure regulating valve for the cupping negative pressure apparatus of the present invention is further improved in that the pressure regulating valve filter trough in the pressure regulating valve body is provided with a filter cotton, and the filter cotton is fixed inside the pressure regulating valve body by a filter cotton fixing cover, air being sucked in passes through the filter cotton and essential oil and body grease in the air are adsorbed by the filter cotton before the air is discharged, and thus impurities are filtered out to maintain normal suction and discharge forces.

### BRIEF DESCRIPTION OF THE DRAWINGS

FIG. 1 is an exploded perspective view of a pressure regulating valve for a cupping negative pressure apparatus of the present invention;

FIG. 2 is a perspective assembled view of the pressure regulating valve for the cupping negative pressure apparatus of the present invention;

FIG. 3 is a cross-sectional exploded view of the pressure regulating valve for the cupping negative pressure apparatus of the present invention;

FIG. 4 is a cross-sectional assembled view of the pressure regulating valve for the cupping negative pressure apparatus of the present invention; and

FIG. 5 is a schematic view showing the use of the pressure regulating valve for the cupping negative pressure apparatus of the present invention.

### DETAILED DESCRIPTION OF THE INVENTION

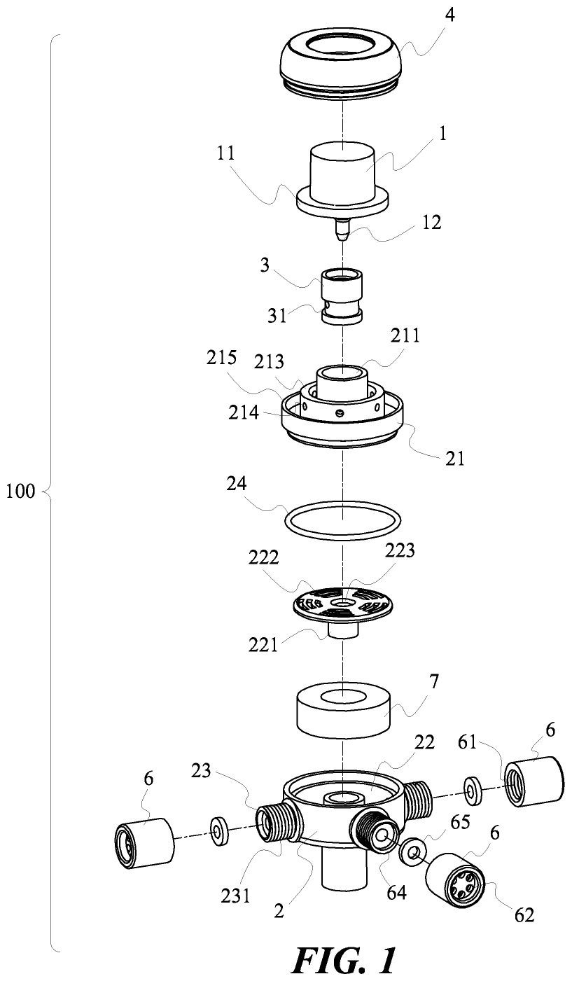

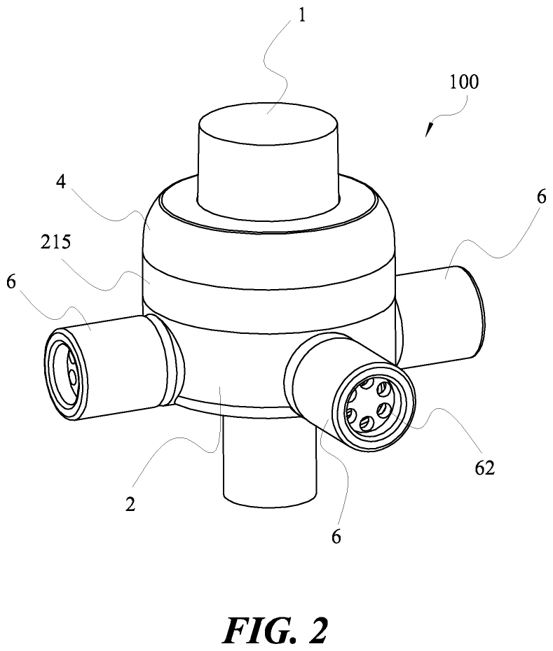

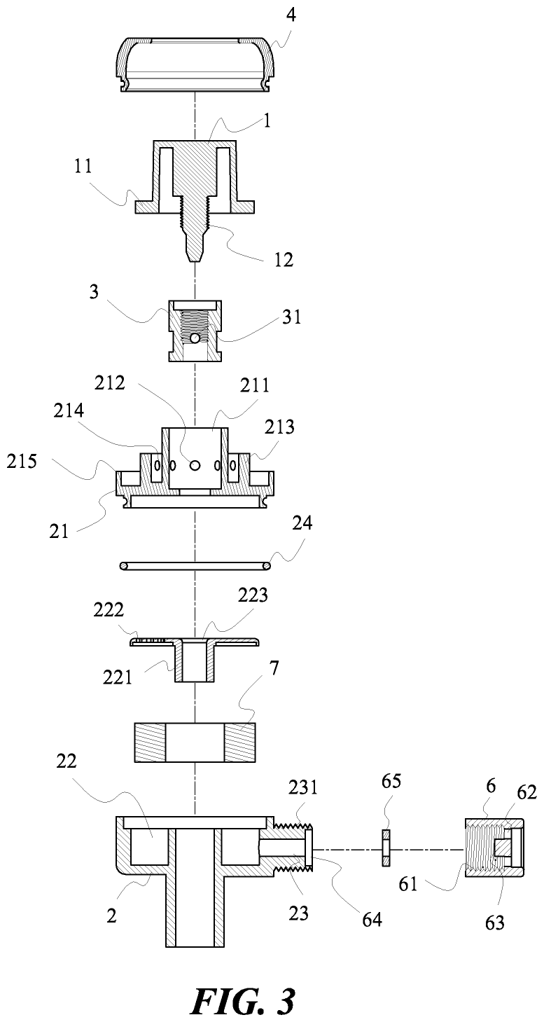

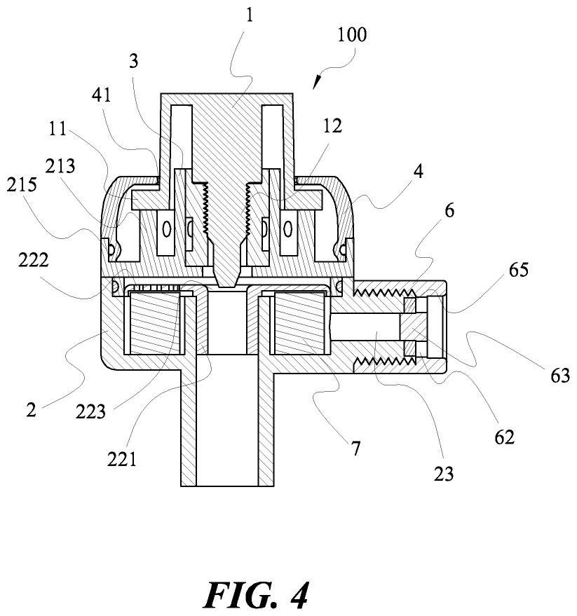

A pressure regulating valve for a cupping negative pressure apparatus of the present invention will be further described below with reference to FIGS. 1 to 5 and specific embodiments. Other advantages and efficacies of the present invention will be readily apparent to those ordinarily skilled in the art from the contents disclosed by the description.

First, please refer to FIG. 1 to FIG. 4 of a pressure regulating valve 100 for a cupping negative pressure apparatus according to the present invention. The pressure regulating valve 100 can be divided into four components, including a pressure regulating valve screw cap 1, a pressure regulating valve body 2, a metal nut structure 3 and a muffler cover 4.

Further, inside the pressure regulating valve body 2 is provided with a pressure regulating valve filter trough 22, and a metal nut structure assembly seat 21 is disposed above the pressure regulating valve filter trough 22, the metal nut structure 3 is tightly mounted in a metal nut structure placement hole 211 in the metal nut structure assembly seat 21 and is not detachable after being placed, and is integrally assembled with the metal nut structure assembly seat 21. The metal nut structure 3 is provided with a plurality of nut pressure relief holes 31 that communicate with circulation holes of placement hole 212 of the metal nut structure placement hole 211.

Further, a user inserts a metal screw structure 12 disposed at a bottom of the pressure regulating valve screw cap 1 into the metal nut structure 3 installed in the metal nut structure assembly seat 21, and rotates the pressure regulating valve screw cap 1 to drive the metal screw structure 12 to rotate tightly into the metal nut structure 3 to cause the pressure regulating valve body 2 in an airtight state.

Further, a muffler cover assembly seat 215 disposed on the metal nut structure assembly seat 21 is capped and assembled with the muffler cover 4 by the user, so that a screw cap brim 11 of the pressure regulating valve screw cap 1 and retaining ring circulation holes 214 of the metal nut structure assembly seat 21 are located in a space between the muffler cover 4 and the muffler cover assembly seat 215 to reduce air noise generated by air flowing to reach inside the pressure regulating valve body 2.

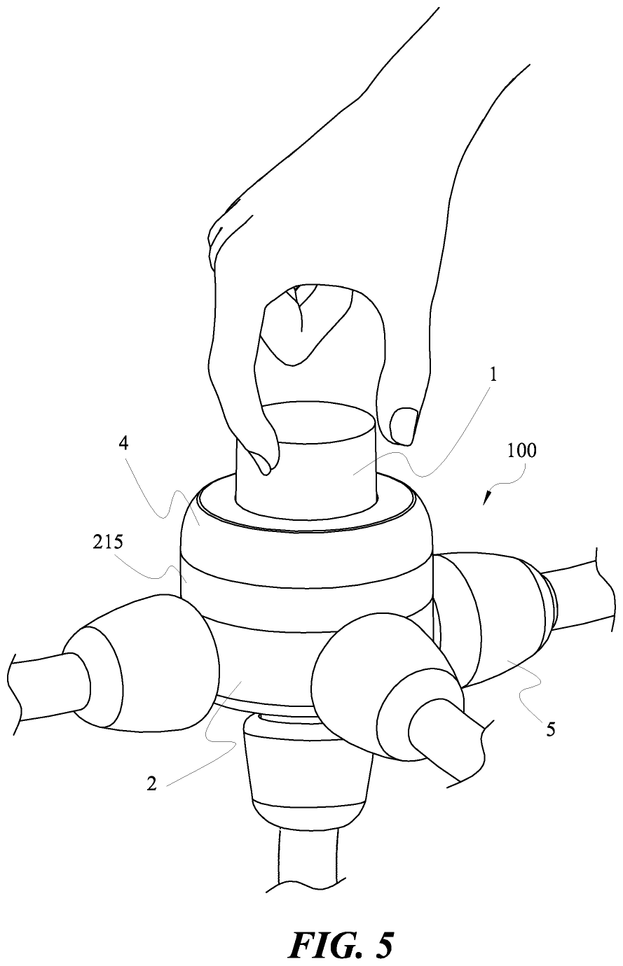

Further, in conjunction with FIG. 5, the user connects communicating tubes 5 in series to negative pressure communication holes 23 of the pressure regulating valve body 2 capped with the muffler cover 4, and other ends of the communicating tubes 5 are connected in series to the negative pressure apparatus (not shown in the figure). Through the negative pressure communication holes 23 around the pressure regulating valve body 2, a same quantity of the communicating tubes 5 can be connected in series, and a plurality of negative pressure cups are connected in series (not shown in the figure), so that the pressure regulating valve 100 is connected between the negative pressure cups and the negative pressure apparatus.

Please refer to FIG. 1 to FIG. 5 as a whole. Further, the user activates the negative pressure apparatus, and the negative pressure apparatus draws the air inside the negative pressure cup out through the communicating tube 5 and the pressure regulating valve 100 to cause a negative pressure state inside the negative pressure cup, and various parts of a human body are covered with the negative pressure cup for performing negative pressure rhythmical massage. The metal screw structure 12 can be driven to rotate upward in the metal nut structure 3 by rotating the pressure regulating valve screw cap 1 during use, and the nut pressure relief holes 31 originally blocked by the metal screw structure 12 are slowly exposed to allow air to circulate from a flow gap 41 between the muffler cover 4 and the pressure regulating valve screw cap 1 to the retaining ring circulation holes 214 disposed at a screw cap retaining ring 213 of the metal nut structure assembly seat 21, then the air flows to the circulation holes of placement hole 212, and finally a small amount of the air flows to reach inside the pressure regulating valve body 2 from the nut pressure relief holes 31; thereby affecting a negative pressure force of the negative pressure apparatus to reach inside the negative pressure cup through the pressure regulating valve 100. The user can control a height of the metal screw structure 12 to rotate upward according to rotation of the pressure regulating valve screw cap 1 to affect an extent of the nut pressure relief holes 31 being exposed, thereby adjusting a magnitude of the negative pressure force inside the negative pressure cup.

Further, after the user finishes using, the muffler cover 4 can be removed, the pressure regulating valve screw cap 1 is rotated and disassembled from the pressure regulating valve body 2, and dirt of the above three components is cleaned and removed to facilitate using the next time.

Further, outside the negative pressure communication holes 23 are capped with quick-plug screw caps 6, and the user can rotate the quick-plug screw cap 6 to adjust whether the negative pressure communication hole 23 is communicated or plugged. The quick-plug screw cap 6 can be operated without having to take it off, avoiding the problem that soft plug used in the conventional technology is easy to get lost.

Further, each of the negative pressure communication holes 23 is provided with a quick-plug screw cap assembly thread 231, an internal thread 61 is disposed inside the quick-plug screw cap 6, so that the quick-plug screw cap 6 can be rotationally assembled on the quick-plug screw cap assembly thread 231 to block the unused negative pressure communication hole 23 in order to prevent air from flowing from the unused negative pressure communication hole 23 to reach inside the pressure regulating valve body 2, which affects negative pressure massage.

Further, the quick-plug screw cap 6 is provided with a plurality of suction and discharge communication holes 62 for communicating with the pressure regulating valve body 2. When the user loosens the quick-plug screw caps 6, the negative pressure communication holes 23 of the pressure regulating valve body 2 are capable of communicating with the suction and discharge communication holes 62 of the quick-plug screw caps 6 for performing negative pressure suction and release massage.

Further, the quick-plug screw cap 6 is internally provided with a quick-plug plug 63, when the quick-plug screw cap 6 is rotated to the tightest, the quick-plug plug 63 blocks the negative pressure communication hole 23 to achieve the effect of plugging.

Further, a quick-plug structure gasket groove 64 is disposed between the quick-plug screw cap 6 and the negative pressure communication hole 23, and a soft anti-pressure relief gasket 65 is disposed in the quick-plug structure gasket groove 64 to prevent a gap from generating when the quick-plug plug 63 plugs the negative pressure communication hole 23 to affect the negative pressure.

Further, a filter cotton 7 is disposed in the pressure regulating valve filter trough 22, and the filter cotton 7 is fixed in the pressure regulating valve body 2 by a filter cotton fixing cover 221, air being sucked in from the negative pressure communication hole 23 passes through the filter cotton 7 and essential oil and body grease in the air are adsorbed by the filter cotton 7 before the air is discharged, thereby preventing suction of the pressure regulating valve 100 from being impaired and reduced.

Further, the metal nut structure assembly seat 21 is detachably disposed above the pressure regulating valve filter trough 22 of the pressure regulating valve body 2, so that the user can separate the metal nut structure assembly seat 21 from the pressure regulating valve filter trough 22, and the pressure regulating valve filter trough 22 can be washed with water or the filter cotton 7 can be replaced.

Further, the filter cotton fixing cover 221 is provided with a plurality of annular holes 222, air first passes through the filter cotton 7 to adsorb essential oil and body grease, the remaining air passes through the annular holes 222 of the filter cotton fixing cover 221, and finally the air is sucked into the negative pressure apparatus through a circular hole 223 in the middle to achieve filtering effect.

Further, a silicone gasket 24 is disposed around the metal nut structure assembly seat 21, after the metal nut structure assembly seat 21 is installed in the pressure regulating valve body 2, the silicone gasket 24 is capable of preventing a gap from forming at an assembly joint.

The above description is only preferred embodiments of the present invention, and is not intended to limit the present invention in any way. Although the present invention has been disclosed above by the preferred embodiments, they are not intended to limit the present invention. Any person having ordinary skill in the art can make slight changes or modifications to the above-disclosed technical contents to become equivalent embodiments without departing from the scope of the technical means of the present invention. Any simple alterations, equivalent changes and modifications made substantially to the above embodiments in accordance with the techniques of the present invention still fall within the scope covered by the technical means of the present invention.

It should be explained that the structures, proportions and sizes depicted in the accompanied drawings are only used to match with the contents disclosed in the specification for understanding and reading by those skilled in the art, and are not intended to restrict the limitation conditions that can be implemented by the present invention, and therefore are not technically significant. Any structural modifications, changes in the proportional relationship or adjustment of sizes without affecting the efficacies generated and the objects achieved by the present invention, should still fall within the scope covered by the technical contents disclosed by the present invention.

While the preferred embodiments of the invention have been set forth for the purpose of disclosure, modifications of the disclosed embodiments of the invention as well as other embodiments thereof may occur to those skilled in the art. Accordingly, the appended claims are intended to cover all embodiments which do not depart from the spirit and scope of the invention.

What is claimed is:

1. A pressure regulating valve for a cupping negative pressure apparatus, characterized in that comprising:
a pressure regulating valve screw cap, a metal screw structure, a muffler cover, a muffler cover assembly seat, a metal nut structure, a pressure regulating valve body, quick-plug screw caps, a filter cotton and a filter cotton fixing cover, inside the pressure regulating valve body being disposed with a metal nut structure assembly seat, the metal nut structure assembly seat being assembled integrally with the metal nut structure, the pressure regulating valve screw cap being assembled integrally with the metal screw structure, through the metal nut structure and metal screw structure the pressure regulating valve screw cap being rotationally assembled with the pressure regulating valve body, rotating the pressure regulating valve screw cap to adjust an air flow amount in the pressure regulating valve body in order to adjust action of negative pressure force, the muffler cover being assembled with the muffler cover assembly seat to cover retaining ring circulation holes on the metal nut structure assembly seat to reduce air noise generated by air flowing to reach inside the pressure regulating valve body, the quick-plug screw caps being capped on negative pressure communication holes provided on the pressure regulating valve body for functioning as air suction and discharge passages, rotating the quick-plug screw cap to adjust whether the negative pressure communication hole being communicated or plugged, the quick-plug screw cap being not required to be removed to avoid getting lost, the filter cotton being disposed in a pressure regulating valve filter trough of the pressure regulating valve body and being fixed inside the pressure regulating valve body by the filter cotton fixing cover, air being sucked in from the negative pressure communication hole passing through the filter cotton and essential oil and body grease in the air being adsorbed by the filter cotton before the air being discharged to prevent suction from being impaired.

2. The pressure regulating valve for the cupping negative pressure apparatus as claimed in claim 1, wherein the pressure regulating valve needs to be connected in series between the cupping negative pressure apparatus and a negative pressure cup during use, the cupping negative pressure apparatus draws the air inside the negative pressure cup out through the pressure regulating valve to generate a negative pressure, and a magnitude of negative pressure force inside the negative pressure cup can be controlled by rotating the pressure regulating valve screw cap.

3. The pressure regulating valve for the cupping negative pressure apparatus as claimed in claim 2, wherein a plurality of nut pressure relief holes are disposed around the metal nut structure inside the metal nut structure assembly seat, the pressure regulating valve screw cap and the pressure regulating valve body are rotationally assembled to be the most tightened, the metal screw structure is capable of blocking the nut pressure relief holes of the metal nut structure so that air is unable to flow into the pressure regulating valve body to cause an airtight state inside the pressure regulating valve body and to maximize the negative pressure force, the pressure regulating valve screw cap is loosened to make the metal screw structure to rotate upward slowly to expose the nut pressure relief holes of the metal nut structure, and to allow a small amount of air to flow to reach inside the pressure regulating valve body from the nut pressure relief holes in order to affect a negative pressure force of the cupping negative pressure apparatus to draw air out from the negative pressure cup, a size of the nut pressure relief holes being exposed is changed according to rotation of the pressure regulating valve screw cap in order to affect an amount of air flowing into the pressure regulating valve body and to adjust the negative pressure force.

4. The pressure regulating valve for the cupping negative pressure apparatus as claimed in claim 3, wherein inside the metal nut structure assembly seat is provided with a metal nut structure placement hole for inserting and assembling the metal nut structure, and the metal nut structure placement hole is disposed with circulation holes of placement hole communicating with the nut pressure relief holes of the metal nut structure, so that air flows smoothly to reach inside the pressure regulating valve body.

5. The pressure regulating valve for the cupping negative pressure apparatus as claimed in claim 4, wherein the metal nut structure assembly seat is provided with a screw cap retaining ring, the pressure regulating valve screw cap is rotated downward to abut against the screw cap retaining ring until unable to be rotated downward anymore, so that the pressure regulating valve screw cap is rotated within a limited range to adjust the negative pressure force, and the screw cap retaining ring is provided with retaining ring circulation holes communicating with the nut pressure relief holes of the metal nut structure inside the pressure regulating valve body for allowing air to flow smoothly to reach inside the pressure regulating valve body.

6. The pressure regulating valve for the cupping negative pressure apparatus as claimed in claim 5, wherein the metal nut structure assembly seat is provided with the muffler cover assembly seat for assembling the muffler cover, the retaining ring circulation holes of the screw cap retaining ring are located in a space between the muffler cover and the muffler cover assembly seat, and air is buffered after circulating from a gap between the muffler cover and the pressure regulating valve screw cap to the space between the muffler cover and the muffler cover assembly seat, and then the air flows to the nut pressure relief holes of the metal nut structure of an innermost layer of the pressure regulating valve body to reduce air noise generated by rapid circulation of air when negative pressure is relieved.

7. The pressure regulating valve for the cupping negative pressure apparatus as claimed in claim 1, wherein the pressure regulating valve body is disposed with the negative pressure communication holes, outside each of the negative pressure communication holes is provided with a quick-plug screw cap assembly thread, an internal thread of the quick-plug screw cap is rotationally assembled on the quick-plug screw cap assembly thread to block the unused negative pressure communication hole in order to prevent air from flowing from the unused negative pressure communication hole to reach inside the pressure regulating valve body to affect negative pressure massage

8. The pressure regulating valve for the cupping negative pressure apparatus as claimed in claim 7, wherein the quick-plug screw cap is provided with a plurality of suction and discharge communication holes for communicating with the pressure regulating valve body, when the user loosens the quick-plug screw cap, the negative pressure communication hole of the pressure regulating valve body is capable of communicating with the suction and discharge communication holes of the quick-plug screw cap for performing negative pressure suction and release massage.

9. The pressure regulating valve for the cupping negative pressure apparatus as claimed in claim 7, wherein the quick-plug screw cap is internally provided with a quick-plug plug, when the quick-plug screw cap is rotated to the tightest, the quick-plug plug blocks the negative pressure communication hole to achieve the effect of plugging.

10. The pressure regulating valve for the cupping negative pressure apparatus as claimed in claim 9, wherein a quick-plug structure gasket groove is disposed between the quick-plug screw cap and the negative pressure communication hole, and a soft anti-pressure relief gasket is disposed in the quick-plug structure gasket groove to prevent a gap from generating when the quick-plug plug plugs the negative pressure communication hole to affect the negative pressure.

11. The pressure regulating valve for the cupping negative pressure apparatus as claimed in claim 1, wherein the metal nut structure assembly seat is detachably disposed above the pressure regulating valve filter trough of the pressure regulating valve body, the metal nut structure assembly seat is separated from the pressure regulating valve filter trough, and the pressure regulating valve filter trough is washed with water or the filter cotton is replaced.

12. The pressure regulating valve for the cupping negative pressure apparatus as claimed in claim 11, wherein the filter cotton fixing cover is provided with a plurality of annular holes, air first passes the filter cotton to adsorb essential oil and body grease, the remaining air passes through the annular holes of the filter cotton fixing cover, and then flows out from the middle to achieve filtering effect.

13. The pressure regulating valve for the cupping negative pressure apparatus as claimed in claim 11, wherein a silicone gasket is disposed around the metal nut structure assembly seat, after the metal nut structure assembly seat is installed in the pressure regulating valve body, the silicone gasket is capable of preventing a gap from forming at an assembly joint.
